(19) United States
(12) Patent Application Publication
(10) Pub. No.: US 2005/0053647 A1
(43) Pub. Date: Mar. 10, 2005

(54) Pharmaceutical product comprising the active substance diamorphine, and its use in a process for treating opiate addiction

(76) Inventors: Rudolf Matusch, Marburg (DE); Bernd Adam, Treysa (DE); Andreas Koch, Melsbach (DE); Hans-Rainer Hoffmann, Neuwied (DE); Bodo Asmussen, Bendorf (DE)

Correspondence Address: PROPAT, L.L.C., 425-C SOUTH SHARON AMITY ROAD, CHARLOTTE, NC, 28211-2841, US

(21) Appl. No.: 10/942,297

(22) Filed: Sep. 16, 2004

Related U.S. Application Data
(63) Continuation of application No. 09/979,413, filed on Feb. 15, 2002.

(30) Foreign Application Priority Data
May 21, 1999 (DE) 199 23 551.1

(51) Int. Cl. A61K 9/70

(52) U.S. Cl. 424/449

(57) ABSTRACT

The invention relates to pharmaceutical preparations that are used in a method for treating opiate addiction or opiate dependence, especially heroin dependence. The active substance used is preferably diamorphine and/or one of the pharmaceutically acceptable acid addition salts thereof. The invention also relates to a method for treating opiate dependence.

### CROSS-REFERENCE TO RELATED APPLICATIONS

This application is a continuation of co-pending U.S. application Ser. No. 09/979,413, filed Feb. 15, 2002. Co-pending U.S. application Ser. No. 09/979,413 is hereby incorporated by reference in its entirety. This application further claims priority to its parent, German patent application no.199 23 551.1 filed May 21, 1999, hereby incorporated by reference herein in its entirety.

### FIELD OF THE INVENTION

The present invention relates to pharmaceutical products for use in a process for treating opiate addiction or opiate dependency, especially heroin dependency. The active substance used is preferably diamorphine (heroin, diacetylmorphine) and/or one of its pharmaceutically acceptable acid addition salts.

### BACKGROUND OF THE INVENTION

The opiates are active substances of the opium poppy (Papaver somniferum). Major opiates include opium, morphine, codeine and heroin. Narcotine, papaverine, narceine, thebaine, laudanosine, xanthaline and noscapine are further ingredients of raw opium. It is only the morphine alkaloids, however, which give rise to a narcotic and analgesic effect.

Frequent use of opiates, especially morphine alkaloids such as heroin, results in a psychological habituation (e.g., escape from everyday reality), accompanied by a physical habituation. If the opiates consumed are withheld, typical withdrawal phenomena occur, particularly including severe withdrawal pains. Opiate dependency or opiate addiction is therefore attributable no longer to the sense of euphoria or alteration of perception desired by the consumer in the initial stage of the addiction, but instead, in particular, to avoidance of the massive pains of abstinence which occur in the case of lack of adequate opiate supply. Although renewed supply of the opiate does blot out the withdrawal pains for a short time, long-term use of opiates is accompanied not only by physical deterioration (pale appearance, outbreaks of perspiration for the slightest reason, gastrointestinal disorders, skin rash, attacks of angina pectoris, disorders of the sexual realm including dysmenorrhea and amenorrhea, reduced potency, gonadic damage, etc.) but also by psychological disorders (irritability, ill humor, depression, etc.) and failure of intellectual performance (memory disorders, loss of concentration, psychotic obsessions, etc.). This leads the addict into isolation from the circle of former acquaintances and, in the long term, into social decline and, not least, into criminality as well.

Heroin dependents have been treated for some time using methadone, a synthetic opoid analgesic. However, methadone is inferior to heroin in therapy, even on intravenous administration, as has been shown by the scientifically evaluated experiment on state-controlled dispensing of heroin which has been ongoing in Switzerland since 1994. There, heroin has been dispensed for intravenous administration or for pulmonary intake, using cigarettes, to 969 selected heroin addicts whose clinical history already included a number of unsuccessful attempts at therapy and who had serious health problems resulting directly or secondarily from heroin addiction. After an initial rise (owing to the deliberately very low initial concentrations), the dose administered could be kept constant from the 6th month on, and in some cases even reduced slightly. Marked improvements in the state of health of the heroin addicts are obtained by state-controlled administration of heroin. In addition, a significant reduction is observed in drug-related crime by heroin addicts. Thus, the benefit to the national economy through the reduction in drug-related crime in Switzerland was 96 francs per person per day.

Intravenous administration of heroin, however, results in severe fluctuations in plasma level. Thus, shortly after an injection, the concentration of heroin in the blood plasma greatly exceeds the minimum level necessary for suppressing the withdrawal phenomenon and enters the toxic range, with severe side effects and confusion. As a result of biotransformation, the plasma level falls back after just a few hours to a concentration below the action threshold, and the unwanted withdrawal phenomena occur.

The use of buprenorphine to treat heroin dependency has also not been successful to date. This may be due to the fact that buprenorphine is very expensive and, as a partial opiate agonist, can cause great problems in cases of possible overdose, since, for example, respiratory depressions induced cannot be treated by administering an antagonist as would normally be used. Furthermore, prolonged use of buprenorphine, as, moreover, is the case with long-term methadone therapy, has exhibited an intensification of the dependency.

Transdermal therapeutic systems for treating dependencies and/or combating addiction are known, but have to date led to commercially available products and some therapeutic successes only in the case of nicotine dependency. Especially in the case of opiate dependency and opiate misuse, and heroin misuse in particular, the hopes placed in lobeline or methadone have not been rewarded.

German Laid-Open Specification DE 196 42 043 A1 proposed the use of acetylmethadol, naltrexone, codeine, dihydrocodeine, and morphine to treat drug dependency or drug addiction. Since none of the last-mentioned active substances, apart from buprenorphine, exceeds the analgesic activity of heroin, these substances are probably limited in the treatment of those most severely dependent on heroin.

Although it appears obvious to choose diamorphine as the active substance with which it might be possible to realize a dose reduction therapy of heroin dependency, transdermal therapeutic systems for the continuous and controlled release of diamorphine for treating the withdrawal symptoms associated with very severe dependency due to heroin addiction are, surprisingly, as yet unknown. This may be a result of the fact that the active substance diamorphine is hydrolyzed relatively easily and therefore it has not yet proven possible to prepare a pharmaceutical product which is intended to release this active substance over a prolonged period and which is not subject to any decomposition even during the time of preceding storage.

Precisely such an administration form, however, is advantageous for constant plasma levels, since a therapeutically sensible concentration corridor is achieved in a controlled manner by means of the route via the skin and by a suitable choice of the size of the permeation area. Constant plasma levels without peaks, which are responsible for side effects and the undersupply associated with severe withdrawal phenomena, however, are the prerequisite for a successful therapy from the standpoint of resocialization, as has been forcefully shown by a large-scale, scientifically evaluated field trial on the state-controlled dispensing of heroin in Switzerland since 1994 (I. Weber, “Verschreibung von Heroin für Drogenabhängige” [Prescription of Heroin for Drug Dependents], Deutsche Apotheker Zeitung, 138, 57, 1998).

It is an object of the present invention to provide a device and a process for treating the abovementioned symptoms which occur as part of the abrupt withdrawal of opiates from an addict, especially for the specific case of heroin addiction. The drug dependent (=addict=patient) is to be taken away from the uncontrolled supply of opiate, with avoidance or reduction of withdrawal symptoms, and finally is to be freed from regular opiate consumption.

A further object is to construct the device in such a way that the active substance it comprises can be dispensed over a prolonged period of time and to ensure that during storage of the device the active substance does not decompose or otherwise lose its activity.

### SUMMARY OF THE INVENTION

This object is achieved by a device capable of continuous and controlled release of an active substance which is preferably diamorphine base or an acid addition salt of diamorphine. The device is suitable for application to the skin or mucous membrane and also for parenteral administration with inclusion of an absorption process. A preferred device is a transdermal therapeutic system (TTS). The construction of a transdermal therapeutic system of this kind is known to the skilled worker. Characteristic features of a TTS are at least one active substance layer and a means of fixing the TTS on the skin, generally a pressure-sensitive adhesive film. Alternatively, the device can be an orally administerable form, e.g., a tablet, a capsule, or a wafer.

### DETAILED DESCRIPTION OF THE INVENTION

In one particular embodiment of the device the active substance, e.g., diamorphine and/or a pharmaceutically compatible acid addition salt of diamorphine, is present in a pharmaceutically compatible aprotic solvent and/or a pharmaceutically compatible solvent having low protolytic [sic] activity. Such pharmaceutically compatible aprotic solvents and solvents having low proteolytic activity include especially N-methylpyrrolidone, R-(+)-limonene, benzyl nicotinate, oleic acid, dimethylisosorbide, lemon oil, Tween 80, and vitamin E.

In one particularly advantageous embodiment, vitamin E and/or vitamin E derivatives are used as an aprotic solvent of this kind or are added in a proportion of at least 5% by weight to the aprotic solvent or to the solvent having low protolytic [sic] activity. It has been found that such combinations of vitamin E with a further pharmaceutically compatible aprotic solvent and/or solvent having low proteolytic activity had a surprisingly positive stabilizing effect on the rate of hydrolytic breakdown of diacetylmorphine. The results of these investigations can be seen from Table 1. This effect is particularly important for the long-term stability of the device.

TABLE 1
Effect of vitamin E on the rate of hydrolytic breakdown* of
diamorphine base

| | Saturation | Diamorphine | Diamorphine |
|---|---|---|---|
| | solubility | content | content |
| Solvent | (in mg/ml) | (in %; t = 0.5 h) | (in %; t = 24 h) |
| N-Methylpyrrolidone | 32.2 | 97.8 | 87.8 |
| R-(+)-Limonene | 25.1 | 96.7 | 87.7 |
| Benzyl nicotinate | 23.2 | 97.8 | 86.5 |
| Oleic acid | 22.6 | 97.4 | 90.3 |
| Dimethylisosorbide | 12.8 | 98.5 | 93.5 |
| Lemon oil | 10.3 | 93.5 | 80.5 |
| Tween 80 | 5.81 | 99 | 93.4 |
| Vitamin E | 1.62 | 100 | 100 |
| N-Methylpyrrolidone/ | 24.8 | 99.8 | 99.7 |
| Vitamin E 1:1 | | | |
| R-(+)-Limonene/ | 19.0 | 99.7 | 99.6 |
| Vitamin E 1:1 | | | |
| Benzyl nicotinate/ | 17.7 | 99.2 | 99.0 |
| Vitamin E 1:1 | | | |
| Oleic acid/ | 17.2 | 99.6 | 99.6 |
| Vitamin E 1:1 | | | |
| Dimethylisosorbide/ | 9.82 | 100 | 99.8 |
| Vitamin E 1:1 | | | |
| Lemon oil/ | 8.00 | 99.1 | 98.9 |
| Vitamin E 1:1 | | | |
| Tween 80/ | 4.45 | 100 | 100 |
| Vitamin E 1:1 | | | |

*the breakdown products quantified were morphine and monoacetyl-morphine (area percentages).

It is further advantageous to use, if desired, during the production process of the device such a pharmaceutically compatible aprotic solvent and/or a pharmaceutically compatible solvent having low proteolytic activity as a solvent or vehicle for the active substance.

In an oral administration form, the aprotic solvents and/or solvents having low proteolytic activity that are used are preferably those which possess a melting point of below 35° C.

In another preferred embodiment of the device, the active substance, especially diacetylmorphine, is in pharmaceutical purity. This means that a degree of purity of 99% relative to the total amount of active substance is achieved. The total amount of unidentifiable extraneous substances is below 1%, with particular preference below 0.1%, based on the active substance.

A further advantage of the device of the invention is the ease of applicability, which permits self-administering. This is of great advantage for therapy not only for the patients but also, in terms of the time burden it avoids, for doctors and/or care staff as well.

In the process for treating opiate addiction, the addict (i.e., the patient) in a first phase is supplied with the active substance in a continuous and controlled manner, the dose administered being adapted initially to the daily requirement of said patient. In this way, first of all, a controlled supply of opiate is obtained which replaces the improper and uncontrolled supply of opiate.

In the second phase of the process, the amount of active substance supplied in a continuous and controlled manner to the patient is then carefully reduced. In this way, a slow, controlled reduction is achieved in the patient's blood morphine level (known as dose reduction treatment). At the end of this second phase, which can if desired be subdivided into a plurality—that is, at least two—stages, it is possible in the best case entirely to abandon further supply of the active substance.

The withdrawal phenomena induced in the case of uncontrolled withdrawal as a result of reduced or abruptly terminated opiate supply, i.e., especially the physical and psychological withdrawal phenomena (e.g., the withdrawal pains), are prevented or reduced in this way. There is likewise an attenuation of the intensity of the phenomena which occur with long-term misuse of opiates, namely the physical wasting, psychological disturbances, and failures of intellectual performance.

An important aspect of the solution is therefore also a sensible administration of the active substance, especially diamorphine, which permits a constant plasma concentration of the active substance or of the principal metabolite, morphine, within a therapeutically sensible concentration corridor over a defined period of time.

Morphines differ from the majority of alkaloids in their more complex molecular structure. Thus the morphines themselves can be seen as derivatives of isoquinoline; alternatively, the phenanthrene skeleton can be regarded as the actual parent ring system of the morphine alkaloids. By varying the basic structure of morphine [(−)-morphine=C₁₈H₂₁NO₃], i.e., by targeted derivatization at certain sites of the molecule, the different features of action can in each case be emphasized. In the case of long-term use of opiates, a habituation effect sets in after a very short time, so that the dose must be increased in order to continue obtaining an effect.

Diamorphine as well, as a semisynthetic opoid, is derived from morphine; it is therefore likewise among the group of the opiates. It is prepared by diacetylation of the phenolic and alcoholic hydroxyl group of the morphine. Substances containing acetyl groups are very susceptible to hydrolysis. By the biotransformation route as well, therefore, diamorphine is broken down to morphine again via monoacetylmorphine; morphine, as the principal metabolite, is therefore the form of diamorphine which is actually active. The hydrolytic breakdown of diamorphine to morphine takes place even in aprotic solvents, albeit not in so dramatic a way as in protic solvents.

Consequently, like hydromorphone and buprenorphine as well, for example, diamorphine (heroin, diacetylmorphine) belongs to the group of part-synthetically prepared morphine derivatives. It possesses the fundamental mode of action of the opiates, including primarily the lowering of the body's reflex reactions to disruptive influences. Therefore, the class of substance has a highly analgesic, antitussive, anxiolytic and antiemetic action. At the same time, however, the morphines also induce constipation and suppress hunger.

The following definitions aid understanding of the invention:

The patients include those persons who are suffering from an opiate addiction and are therefore dependent on a regular, essentially uncontrolled supply of opiate. The terms “addiction”, “dependency” and “misuse” and the like should be regarded as synonymous for the purposes of the present description, despite the fact that these terms are occasionally defined differently in technical circles. The nature of the drug misuse which the present invention is intended to treat is, however, drug dependency of the opiate type. The opiate dependent (e.g., heroin dependent) becomes the patient as soon as he or she decides to undergo a drug addiction treatment.

The addiction-causing opiates in question are those whose long-term improper consumption gives rise to dependency, i.e., morphine alkaloids such as heroin, morphine, opium or cocaine, and also combinations of these substances with one another or with other intoxicants or narcotics (such as alcohol, nicotine, amphetamines, cannabis, barbiturates, etc.).

In patients who wish to undergo drug addiction treatment, sudden interruption of the opiate supply is accompanied by withdrawal phenomena, both physical and psychological. These phenomena include a particular yearning for the opiate, referred to as “craving”, depression and depressive moods, irritability, loss of drive, lack of motivation, loss of appetite and altered dietary habits, nausea, shaking, unease, psychomotor alterations, and irregular sleeping behavior.

The device for controlled and continuous release comprises an active substance. The active substance comprises derivatives of isoquinoline and/or derivatives of phenanthrene. By this are meant, inter alia, morphine alkaloids, i.e., derivatives of (−)-morphine and/or pharmaceutically compatible acid addition salts of the derivatives of morphine. The preferred active substance is diamorphine, present as diamorphine base and/or in the form of a pharmaceutically compatible acid addition salt of diamorphine. Alternatively, the active substance can be a combination of at least one derivative of (−)-morphine with underivatized, i.e., chemically unaltered, (−)-morphine. Finally, the active substance can also be present in the form of an inclusion compound in, for example, cyclodextrins, and/or adsorbed on ion exchange resins.

The pharmaceutically compatible acid addition salts of the active substance include the salts which form when the basic centers of the morphine alkaloids react with appropriate acids. Appropriate acids include hydrochloric acid, sulfuric acid, hydrogen bromide, lactic acid, formic acid, propionic acid, acetic acid, oleic acid, phosphoric acid, citric acid, ascorbic acid, and tartaric acid. Pharmaceutically compatible acid addition salts formed in this case are hydrochlorides, sulfates and hydrogen sulfates, hydrobromides, iodides, lactates, acetates, formates, propionates, oleates, phosphates and hydrogen phosphates, citrates, ascorbates, and tartrates. Preferred pharmaceutically compatible acid addition salts of diamorphine are diamorphine hydrochloride, diamorphine hydrogen sulfate, diamorphine tartrate, diamorphine citrate, diamorphine acetate, diamorphine lactate, and diamorphine hydrobromide.

The device can possess the form of a solution, suspension, emulsion, foam, implant, ointment, paste, suppository, plain tablet, powder, coated tablet, spray, or aerosol. The preferred form of the device is a transdermal therapeutic system.

The device is applied to the skin or mucous membrane of the patient. In other words, the modes of administration are transdermal, mucosal, buccal, lingual, sublingual, enteral (=oral), intestinal, nasal, rectal, and inhalative. Where the device is a solution or an implant, administration may also take place parenterally into the interior of the patient's body. This mode of administration, however, takes place exclusively with inclusion of an absorption process, i.e., intracutaneously, subcutaneously, intramuscularly, or intraperitoneally.

The device has the further capacity for controlled and continuous release of the active substance. By this is meant that the active substance is released at the site of administration over a prolonged period of time. Appropriate sites of administration include the undamaged skin, the oral, lingual, nasal, gastric, intestinal and rectal mucosae, and also the bronchial and alveolar epithelium. In the case of parenteral administration with inclusion of an absorption process, the skin, subcutis, muscles, and abdominal cavity are appropriate sites of administration. In each case, the active substance is released in a controlled, i.e. temporally retarded, manner.

Said prolonged period of time is a period of time lasting at least several hours. The periods of time in question are at least about 6, 12 or 16 hours. Preference is given, however, to periods of time of about 24, 48 or 72 hours. In the case of an implant, the prolonged period of time may even extend to at least about 3 to 7 days, but preferably at least about 14 days to about 3 months. Where the device is a transdermal therapeutic system, preferred periods of time are about 16, 24, 48 or 72 hours.

The pharmaceutically compatible aprotic solvents and/or pharmaceutically compatible solvents having low protolytic [sic] activity that are preferably used as solvents or vehicles during the production of the device include N-methylpyrrolidone, benzyl nicotinate, R-(+)-limonene, lemon oil, oleic acid, undecenoic acid, dimethylisosorbide, polyoxeyethylene sorbitan monolaurate, polyoxyethylene sorbitan monooleate, polyoxyethylene sorbitan monopalmitate, polyoxyethylene sorbitan monostearate, polyoxyethylene sorbitan trioleate, polyoxyethylene sorbitan tristearate (which are also present in the products known under the tradename Tween 20, 40, 60, 80 or 85), and combinations thereof.

A particularly preferred solvent or vehicle for the active substance is vitamin E, i.e., (+)-_{C}-tocopherol, DL-_{C}-tocopherol and derivatives of vitamin E such as vitamin E acetate and vitamin E succinate.

The process for treating opiate addiction, which involves using the device, can be subdivided into two stages. The duration of the process depends on the severity of the opiate addiction. It is clear that “round-the-clock” supply with the active substance is particularly advantageous, since said withdrawal symptoms may sometimes occur just a few hours after the last administration of the addiction-causing opiate.

In the first phase of the process for treating opiate addiction, the patient is supplied the active substance in a continuous and controlled manner, the dose administered daily being initially adapted to the existing daily requirement of said patient. At the same time, improper—that is, uncontrolled—opiate supply is avoided completely. The duration of this first phase of the process can amount to several days or weeks.

In the second phase of the process, which may be divided if appropriate into a plurality of stages, the amount of active substance supplied continuously and controlledly to the patient is carefully reduced. This means that the dose administered daily is slightly below the patient's daily requirement hitherto. Such a dose is administered in the first stage over a period of several days or weeks. In further stages, in turn, the dose administered daily is reduced in each case to below the level of the dose administered daily in the immediately preceding stage. In this way, the daily dose of the active substance is reduced in stages, it being possible for each stage to extend over a period of several days or weeks. An exact therapeutic plan, with precise indications of the dose to be administered each day in these stages, can, however, only be drawn up by the doctor individually for each patient in accordance with the severity of his or her opiate dependency.

In this way, a slow and controlled reduction in the patient's blood morphine level is obtained (known as dose reduction treatment). At the end of this second phase, further supply of the active substance may be abandoned completely. The entire therapy may extend over several weeks or months.

In the context of such a therapy, a device intended for once-a-day release of the active substance, i.e. capable of continuous controlled release of the active substance over 24 hours, is replaced once a day by a new device of this kind. If using a device intended for two- or three-day administration, then in the context of the therapy, accordingly, this device need be replaced by a new device only every second or third day, respectively.

In the context of such a therapy a device of the invention is used which releases the active substance in a controlled and continuous manner. Where the device to be used is a transdermal therapeutic system which comprises the active substance and which releases said active substance in a controlled manner over 16 h, said device is, for example, applied in the morning to undamaged skin, worn through the day for about 16 hours, and removed before the patient goes to bed. On the next morning, the next 16-hour TTS is applied. When using a transdermal therapeutic system which comprises the active substance and which releases said active substance in a controlled manner over 24 h, said device is, for example, applied in the morning to undamaged skin, worn through the day and in the subsequent night, and replaced the next morning by the next 24-h TTS. In accordance with the objective of staged reduction of the active substance dose supplied daily in the second phase of the process, a device of the invention which releases a smaller amount of the active substance is used at the beginning of each new stage.

When a device of the invention is used which releases the active substance in a continuous and controlled manner over an even longer period of time, said device is replaced by new devices at appropriate intervals. In one particular embodiment the beginning of a new stage of the second phase, in which the daily dose administered is reduced relative to the dose administered daily in the preceding stage, may coincide with the administration of a new device with reduced release of active substance. This can be done, for example, when using an implant.

In every case, the devices of the invention achieve a constant plasma level during the first phase and during each stage of the second phase. In this way it is ensured that there is a controlled concentration of morphine in the blood of the patient throughout the duration of the process.

The therapy, i.e., the process of the invention, may if desired be conducted, for some of the time at least, with the addition of circulation-promoting medicaments, vitamins, tranquilizers, etc. It is likewise of advantage to support the therapy by additional psychological care.

Specific embodiments of the device of the invention are described in the text below:

In one oral formulation the active substance is encapsulated in a semipermeable membrane which comprises, for example, cellulose acetate. A drill or laser is used to bore a small aperture in the capsule material. In the body of the patient to be treated, following intake of the device, water is absorbed through the capsule material. The active substance is then released externally through the small aperture in the desired continuous and controlled manner by virtue of osmotic pressure. Systems of this kind are described, for example, in U.S. Pat. No. 3,760,805 and U.S. Pat. No. 3,987,790. In these systems, the pharmaceutical active substances can be present in solid form or adsorbed on ion exchange resins.

In an (orally administerable) mucoadhesive embodiment of the device, the active substance is incorporated in a bioadhesive, biocompatible, water-soluble and/or at least water-swellable polymer matrix. A polymer matrix of this kind may, for example, comprise polyacrylic acid carboxymethylcellulose [sic], and further “water-swellable polymers” known from EP 421 454 A. The construction of the mucoadhesive devices can be similar to that of transdermal systems, except that the release of active substance can take place in two directions, i.e., both in the direction of the mucosa and in the reverse direction (i.e., into the body cavity, e.g., stomach, intestine, etc.) The construction of various mucoadhesive systems is described at Ahuja, Khar, Ali in Drug Development and Industrial Pharmacy, 23(5), 489-515 (1997).

A transdermal therapeutic system is a multilayer-constructed pharmaceutical administration form. It comprises a backing layer, which is impervious to the active substance or substances, and a pressure-sensitive adhesive layer, which comprises the active substance(s). There are also TTS types where the release of the active substance from a reservoir is controlled by a semipermeable or microporous membrane. Basic TTS types are described in detail by Y. W. Chien in “Developmental Concepts and Practice in Transdermal Therapeutic Systems” in Y. W. Chien, Transdermal Controlled Systemic Medications, Marcel Dekker Inc., New York (1982), Chapter 2, pp. 25-81. In order to avoid repetition, the relevant content of said chapter is incorporated by reference as part of the disclosure of this invention.

The TTS matrix of the present invention comprises preferably water-insoluble pressure-sensitive adhesives, based for example on polyacrylate, polymethacrylate, polyisobutylene, silicone, styrene/butadiene copolymer or styrene/isoprene copolymer, particularly preferred adhesive matrices comprising polymers based on acrylate and/or methacrylate, especially acrylate copolymers of 2-ethylhexyl acrylate, vinyl acetate, acrylic acid and glycidyl methacrylate with or without titanium chelate ester.

Further embodiments of suitable transdermal therapeutic systems are described in German Patent DE 33 15 272 (corresponding to U.S. Pat. No. 4,769,028). This system consists of an impermeable cover layer, a specially constructed supersaturated active substance reservoir comprising a polymer matrix, which is connected to said cover layer, a pressure-sensitive adhesive layer which is permeable for the active substance and is connected to the reservoir, and a protective layer which covers the pressure-sensitive adhesive layer and is removable for use. Also possible are systems in which the inherent tackiness of the reservoir layer is so high that it simultaneously constitutes the pressure-sensitive adhesive layer. German Patent DE 38 43 239 (corresponding to U.S. Pat. No. 5,089,267) describes such a system.

Other suitable transdermal devices are described in U.S. Pat. No. 3,742,951, U.S. Pat. No. 3,797,494, U.S. Pat. No. 3,996,934 and U.S. Pat. No. 4,031,894. These TTS types consist basically of a backing layer, which represents one of the surfaces, an adhesive layer permeable for the active substance, which represents the other surface, and, finally, a reservoir, which contains the active substance between the two layers forming the surface. Alternatively, the active substance can also be contained in a large number of microcapsules which are distributed in the permeable adhesive layer. In every case, the active substance is released continuously from the reservoir or the microcapsules, through a membrane, into the adhesive layer permeable for the active substance, which is in contact with the skin or mucosa of the patient. In the case of microcapsules, the capsule material may also act as a membrane.

The concentration of the active substance depends on the size of the active substance release area of the reservoir. For instance, the active substance content can amount to from 0.1 to 50% by weight of the polymer matrix. Particular preference is given to an active substance content of from 10 to 15% by weight.

Preferred transdermal therapeutic systems possess a layer having an active substance content of from 3 to 25% by weight. Particular preference is given to an amount of from 10 to 20% by weight of, for example, diamorphine and/or a pharmaceutically compatible acid addition salt of diamorphine.

In the layer containing the active substance, the transdermal therapeutic systems of the invention may further comprise at least one solvent or vehicle for the active substance.

The matrix of the TTS of the invention may further comprise vitamin E or a vitamin E derivative. In one preferred embodiment this amount is 5-15% by weight, based on the overall weight of the polymer matrix. In another preferred, variant embodiment of the invention, the vitamin E content of the polymer matrix is 6.25% by weight, in the form of an oily solution of D-_{C}-tocopherol. The addition of vitamin E or said derivatives, moreover, reduces the solubility of the active substance in the matrix material. This in turn promotes a greater skin flux, since the thermodynamic activity of the active substance in the polymer matrix is raised.

The release area of the active substance matrix can be varied as a result of the fixed assembly with a backing film (“backing layer”). This film, which in one preferred embodiment is impermeable to water vapor, can consist, for example, of polyester, polypropylene or coated paper, with a thickness of approximately 10-100 μm. In one preferred embodiment the backing layer consists of polyethylene terephtalate (PET) with a layer thickness of from 10 to 50 μm, it being possible for the PET to be clear, opaque, or printed.

Optionally, the TTS may also be equipped with a so-called over-patch or “cover patch” made from textile fabric in order to achieve additional fixing to the skin in the case, for example, of heavy perspiration.

Furthermore, the TTS has the characteristic feature of a removable protective layer (“release liner”) having a layer thickness of from about 40 to about 100 μm. The removable protective layer, which is in contact with the reservoir layer and must be removed from the TTS before use, consists, for example, of the same materials as used to produce the backing layer. The redetachability is brought about, for example, by treating the film surfaces with silicone. Optionally, in addition to the siliconization, the protective layer may have been metalized by means, for example, of vapor deposition of aluminum. The protective layer may further be provided with application aids by means of which it can be removed more easily from the TTS. The simplest form of application aid is a projection of the protective layer format relative to the active substance matrix format. Another conceivable application aid is the punching through of different areal segments of the protective film. In one preferred embodiment the protective layer consists of polyethylene terephtalate (PET) having a layer thickness of about 100 μm, it being possible for the PET again to be clear, opaque, or printed.

One advantage of the device are the particularly low costs of the therapy. One reason for this is that it is not necessary for skilled staff (doctors, nurses) to carry out the application of the device. Furthermore, the devices described are less expensive to produce than comparable devices for parenteral administration without absorption, i.e., for instance, infusion bottles and injection syringes.

A further advantage of the devices described is the difference between them and the smoking or injection that is customary in heroin misuse. On the one hand, consequences of long-term injection, such as abscesses or other venal disorders, or the risk of embolism, is precluded; on the other hand, infections such as HIV or hepatitis, which may occur in cases of improper injection, are ruled out.

For this application form of the active substance, especially in the case of diamorphine in a TTS, an additional advantage is the safety aspect with respect to possible misuse. In contradistinction to the diamorphine administration forms employed to date, improper use of the device is virtually impossible, since without expert knowledge and complex laboratory equipment the extraction and isolation of the active substance from a TTS matrix is not feasible.

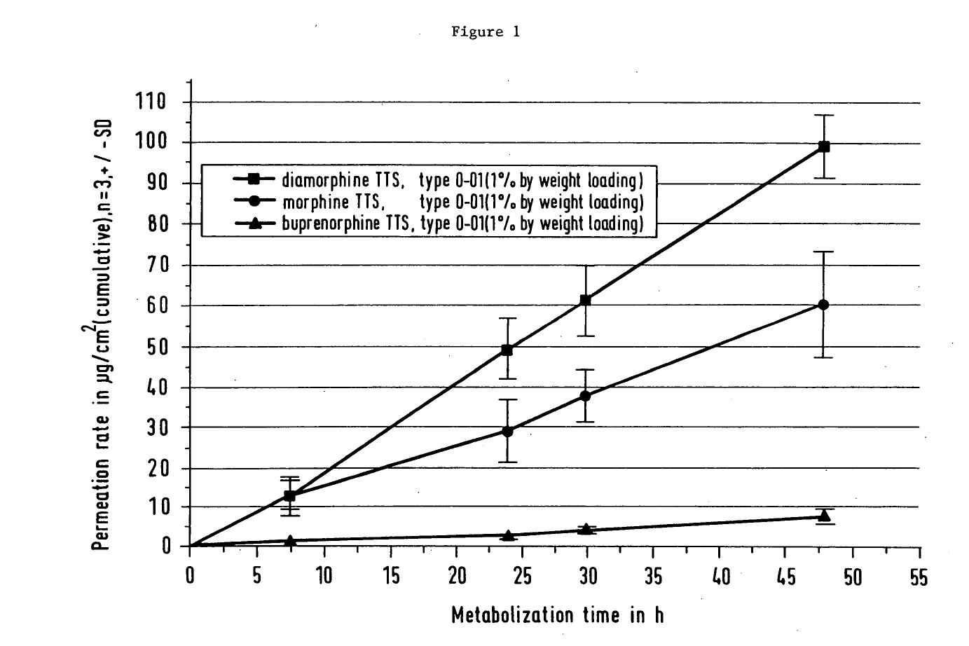

FIG. 1 shows a comparison of skin permeation (nude guinea pig) of various opiate bases from a TTS of identical formulation (released in citrate buffer pH=3.0+0,1% NaN₃ at T=37° C.). Diamorphine is determined as total of heroin+morphine metabolite.

The figure shows the better skin passage of diamorphine in relation to morphine and buprenorphine, under controlled release in the in vitro skin model of the nude guinea pig, from a matrix system with the same base formulation and loading. The skin flux in flow equilibrium (permeation rate in cg/cm² _{C}h) extends at least up to time t=48 h and in some cases is also markedly higher than that of morphine and buprenorphine. This demonstrates the outstanding continuous and controlled transdermal administration of the active substance for treatment, in the sense of the present invention, for heroin addiction.

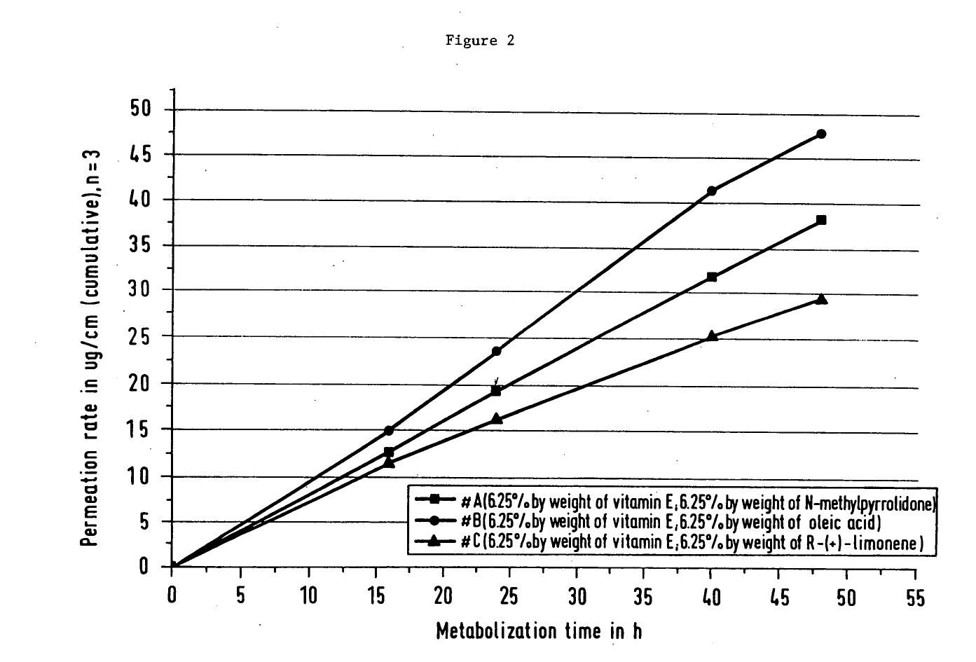

FIG. 2 shows a comparison of skin permeations (human complete skin, ID 446) of various TTS of diamorphine base based on polyacrylate (active substance content=10.0% by weight).

The figure shows the permeation behavior of TTS formulations of the invention under in vitro conditions at 37° C. on the human skin diffusion model. For this purpose, TTS having a permeation area of 1.54 cm² were produced, were bonded to the surface of sectioned samples of complete human skin (cosmetic operation material from female breast reductions excluding subcutaneous fatty tissue), and investigated by means of HPLC for their permeation rates (dependency of the concentration of permeated active substance as a function of time in the acceptor medium) in modified FRANZ diffusion cells. The acceptor used was 0.9% strength sodium chloride solution with the addition of 0.1% sodium azide. The TTS were of the same base formulation and active substance loading. They differed only in the selection of the solvent and/or plasticizer. Since the skin flux (permeation rate in μg/cm² _{C}h) in the steady state (flow equilibrium) extends at least up to time t=48 h, the prerequisite for controlled transdermal application via the skin, for the purpose of treatment of heroin addiction, is met.

The examples below serve to illustrate the production of devices of the invention:

### EXAMPLE 1

### Production, and the Formulation Constituents, of a Transdermal Therapeutic System of the Invention

0.3125 g of D-_{C}-tocopherol (corresponding to 6.25% by weight) was placed in a stirred vessel and dissolved by adding 0.3125 g of N-methylpyrrolidone (corresponding to 6.25% by weight). 0.5 g of diamorphine base (corresponding to 10% by weight) in portions was introduced in turn into this solution, with stirring. With the addition of 1 ml of ethyl acetate, stirring was continued until the solid material had dissolved completely (approximately 15 minutes, visual monitoring). This solution was then stirred in portions into 10.39 g of a self-crosslinking acrylate copolymer of 2-ethylhexyl acrylate, vinyl acetate, acrylic acid and glycidyl methacrylate (37.3% by weight in a solvent mixture of 54:35:11 ethyl acetate:2-propanol:hexane; DuroTak 1753 from National Starch, Neustadt/Weinstrasse, Germany).

The batch was subsequently stirred at room temperature for about 30 minutes and aftertreated in an ultrasound bath at T=40° C. for 15 minutes in order to remove excess air from the composition. The adhesive solution was then coated in a wet-film thickness of 300 μm onto a siliconized polyethylene terephtalate film (Hostaphan RN 100 54B/54B from Hoechst, Frankfurt, Germany) using an appropriate coating bar. After the solvents had been removed by drying at 50° C. for 30 minutes, the adhesive film was lined by lamination with a 15 μm polyester film. Using appropriate cutting tools, the intended application areas were punched out and the edges removed by lattice stripping.

### EXAMPLE 2

### Production and Formulation Constituents of a Further Transdermal Therapeutic System of the Invention

Example 1 was repeated but using equal parts by weight of oleic acid instead of N-methylpyrrolidone.

### EXAMPLE 3

### Production and Formulation Constituents of a Further Transdermal Therapeutic System of the Invention

Example 1 was repeated but using equal parts by weight of R-(+)-limonene instead of N-methylpyrrolidone.

1. A pharmaceutical device for continuous and controlled release of at least one active substance for application to the undamaged skin, to the oral, lingual, nasal or rectal mucosae, to the bronchial or alveolar epithelium, or parenterally with inclusion of an absorption process, wherein the active substance is diamorphine, which is present as diamorphine base, in the form of a pharmaceutically compatible acid addition salt from the group consisting of the hydrochloride, the sulfate, the hydrogen sulfate, the hydrobromide, the iodide, the lactate, the acetate, the formate, the propionate, the oleate, the phosphate, the hydrogen phosphate, the citrate, the ascorbate and the tartrate of diamorphine or in the form of an inclusion compound.

2. The device as claimed in claim 1, which possesses the form of a solution, suspension, emulsion, implant, suppository, plain tablet, powder, coated tablet, transdermal therapeutic system, wafer, spray, or aerosol.

3. The device as claimed in any of claims 1 to 2, wherein said active substance further comprises (−)-morphine and/or a pharmaceutically compatible acid addition salt of (−)-morphine.

4. The device as claimed in any of claims 1 to 3, which possesses the form of a multilayer-constructed transdermal therapeutic system having a layer comprising a pressure-sensitive adhesive.

5. A transdermal therapeutic system as set forth in claim 4, wherein said pressure-sensitive adhesive comprises polymers selected from the group consisting of polymers based on acrylate and/or methacrylate, silicones, polyisobutylenes, styrene/butadiene copolymers, styrene/-isoprene copolymers, and the esters of hydrogenated rosin.

6. A system as claimed in claim 5, wherein the polymers present in the pressure-sensitive adhesive are self-crosslinking acrylate polymers based on acrylate and/or methacrylate.

7. The system as claimed in claim 6, which comprises 2-ethylhexyl acrylate, vinyl acetate and acrylic acid in the monomer composition of the self-crosslinking acrylate polymer.

8. The system as claimed in claim 4, wherein one layer comprises from 0.1 to 50% by weight of the active substance and/or of a pharmaceutically compatible acid addition salt.

9. The system as claimed in claim 4, which further comprises a substance selected from the group consisting of N-methylpyrrolidone, benzyl nicotinate, R-(+)-limonene, lemon oil, oleic acid, undecenoic acid, dimethylisosorbide, polyoxeyethylene sorbitan monolaurate, polyoxyethylene sorbitan monooleate, polyoxyethylene sorbitan monopalmitate, polyoxyethylene sorbitan monostearate, polyoxyethylene sorbitan trioleate, and polyoxyethylene sorbitan tristearate.

10. The system as claimed in claim 4, wherein the layer comprising the active substance further comprises from 1 to 15% by weight of vitamin E and/or a vitamin E derivative.

11. A process for producing a pharmaceutical device comprising as active substance diamorphine, which comprises using a pharmaceutically compatible aprotic solvent and/or a pharmaceutically compatible solvent having low protolytic [sic] activity as a solvent or vehicle for said active substance.

12. A process for producing a pharmaceutical device as claimed in claim 4, which comprises the steps of:
a) introducing the active substance into the solution or melt of a polymeric vehicle,
b) coating this polymer solution or polymer melt, containing active substance, onto a carrier web,
c) solidifying the coated polymer solution or polymer melt, containing active substance, by removing the solvent or solvents, by cooling or by leaving it to stand, with or without crosslinking of the polymer and
d) punching individual patches from the polymer composition, containing active substance, that is obtained after said solidifying.

13. The use of a derivative of (−)-morphine and/or of a pharmaceutically compatible acid addition salt of a derivative of (−)-morphine for producing a medicament for use in the continuous and controlled administration of the active substance in a process for treating psychological and/or physical withdrawal symptoms associated with the sudden termination of opiate misuse.

14. A process for disaccustoming a patient dependent on uncontrolled supply of opiate, which comprises
a) a first phase, in which an active substance dose adapted to the daily opiate requirement of the patient is administered daily in a continuous and controlled manner, and
b) a second phase, which comprises at least one stage, said at least one stage comprising daily administration in a continuous and controlled manner of an active substance dose below the dose administered to the patient in the first phase.

15. The process as claimed in claim 15, wherein said second phase includes at least one further stage, comprising daily administration, in a continuous and controlled manner, of an active substance dose below the dose administered to the patient in the preceding stage.
